(19) United States
(12) Patent Application Publication
(10) Pub. No.: US 2009/0292177 A1
(43) Pub. Date: Nov. 26, 2009

(54) Method and apparatus for positioning a tissue recovery instrument in confronting adjacency with a target tissue volume

(76) Inventors: Philip E. Eggers, Dublin, OH (US); Eric A. Eggers, Columbus, OH (US); Andrew R. Eggers, Ostrander, OH (US)

Correspondence Address: MUELLER AND SMITH, LPA;MUELLER-SMITH BUILDING, 7700 RIVERS EDGE DRIVE, COLUMBUS, OH, 43235, US

(21) Appl. No.: 10/235,131

(22) Filed: Sep. 5, 2002

Related U.S. Application Data
(63) Continuation-in-part of application No. 09/904,396, filed on Jul. 12, 2001, now Pat. No. 6,471,659.
Application No. 09/904,396 is a continuation-in-part of application No. 09/472,673, filed on Dec. 27, 1999, now Pat. No. 6,277,083.

(51) Int. Cl. A61B 1/32; A61B 18/14

(52) U.S. Cl. 600/219; 606/41

(57) ABSTRACT

A target tissue volume is accessed with a cannular instrument, the tip surface of which supports a precursor electrode assemblage which is electrosurgically excitable. The instrument tip initially is inserted through an incision made in the skin of a patient utilizing a pair of retractor components, the tips of which are located at a proper depth for positioning the precursor electrodes of the recovery instrument. The retractor components also are configured to define a guidance channel for receiving the tip of the recovery instrument. By stabilizing the instrument when the precursor electrodes are adjacent the tips of the retractor components and then slidably removing the retractor apparatus along the surface of the instrument, the tissue is “set” to assure proper precursor electrode positioning. The precursor electrodes are configured to exhibit an equivalent diameter having a value of at least about 90% of the diameter of the recovery instrument and are spaced forwardly of the tip surface of the instrument a distance for enhancing instrument maneuvering.

### CROSS-REFERENCE TO RELATED APPLICATIONS

The present application is a Continuation-in-Part of application Ser. No. 09/904,396 filed Jul. 12, 2001 entitled “Minimally Invasive Intact Recovery Of Tissue” by Eggers, et al. which, in turn, is a Continuation-in-Part of application Ser. No. 09/472,673, filed Dec. 27, 1999, now U.S. Pat. No. 6,277,083 by Eggers, et al., issued Aug. 21, 2001 and entitled “Minimally Invasive Intact Recovery Of Tissue”.

### STATEMENT REGARDING FEDERALLY SPONSORED RESEARCH

Not applicable.

### BACKGROUND OF THE INVENTION

Developments of the diagnosis of tumorous cancer and its subsequent treatment continues to somewhat rapidly evolve. These developments particularly have been apparent in connection with cancer of the breast, perhaps in consequence of an estimation that one out of eight women will face cancer at some point in her life.

Among the developments, techniques for detection with imagining devices have permitted the identification of suspect tumor of relatively small size, for example, 5 mm or smaller. Such imaging has nurtured a concomitant development of biopsy and target tissue removal systems.

The historic biopsy option available upon detection of a suspect tumor is an open surgical biopsy or excisional biopsy. Prior to surgery, a radiologist, using mammography, inserts a wire into the breast to locate the tumor site. Later during surgery, the surgeon makes an incision in the breast and removes a large section of breast tissue, including the suspect tissue and a margin of healthy tissue surrounding the tumor. As with other similar procedures, such as those described above, open surgery may result in high levels of blood loss, scarring at the location of the incision and permanent disfigurement, due to the removal of relatively large amounts of tissue. Because of the critical prognostic significance of tumor size, the greatest advantage of the excisional biopsy is that the entire area of the suspect tumor is removed. After being removed and measured, the specimen is split by a pathologist in a plane that should bisect a tumor if present, then the margin between tumor and healthy tissue is examined. Microscopic location of carcinoma near the margin provides information for future prognosis. Thus the pathology laboratory is oriented to the morphological aspect of analysis, i.e. the forms and structures of involved tissue.

For information on pathology of breast biopsy tissue, see:
(1) Rosen, Paul Peter. Rosen's Breast Pathology.

Philadelphia: Lippincott-Raven Publishers, 1997. 837-858.

Other less invasive options are available which avoid the disadvantages associated with open surgery. One such less-invasive option is that of needle biopsy, which may be either fine needle aspiration or large core. Fine needle aspiration (FNA) is an office procedure in which a fine needle, for example of 21 to 23 gauge, having one of a number of tip configurations, such as the Chiba, Franzeen or Turner, is inserted into the breast and guided to the tumor site by mammography or ultra sound imaging. A vacuum is created and the needle moved up and down along the tumor to assure that it collects targeted cellular material. Generally, three or more passes will be made to assure the collection of a sufficient sample. Then, the needle with the tissue sample is withdrawn from the breast.

The resulting specimen is subject to a cytologic assay, as opposed to the above-noted morphological approach. In this regard, cell structure and related aspects are studied. The resultant analysis has been used to improve or customize the selection of chemotherapeutic agents with respect to a particular patient.

While a fine needle aspiration biopsy has the advantages of being a relatively simple and inexpensive office procedure, there are some drawbacks associated with its use. With fine needle aspiration, there is a risk of false-negative results, which most often occur in cases involving extremely fibrotic tumor. In addition, after the procedure has been performed there may be insufficient specimen material for diagnosis. Finally, with fine needle aspiration alone the entire area of suspect tissue is not removed. Rather, fragmented portions of tissue are withdrawn which do not allow for the same type of pathological investigation as the tissue removed during an open surgery biopsy.

This limitation also is observed with respect to large core needle biopsies. For a large core needle biopsy, a 14 to 18 gauge needle is inserted in the breast having an inner trocar with a sample notch at the distal end and an outer cutting cannula. Similar to a fine needle aspiration, tissue is drawn through the needle by vacuum suction. These needles have been combined with biopsy guns to provide automated insertion that makes the procedure shorter and partially eliminates location mistakes caused by human error. Once inserted, multiple contiguous tissue samples may be taken at a time.

Samples taken during large core needle biopsies may be anywhere from friable and fragmented to large pieces 20 to 30 mm long. These samples may provide some histological data, unlike fine needle aspiration samples, however, they still do not provide the pathological information available with an open surgical biopsy specimen. Further, as with any mechanical cutting device, excessive bleeding may result during and following the procedure. Needle biopsy procedures are discussed in:
(2) Parker, Steve H. “Needle Selection” and “Stereotactic Large-Core Breast Biopsy.” Percutaneous Breast Biopsy. Eds. Parker, et al. New York: Raven Press, 1993. 7-14 and 61-79.

A device which is somewhere between a needle biopsy and open surgery is referred to as the Advanced Breast Biopsy Instrumentation (ABBI). With the ABBI procedure, the practitioner, guided stereotacticly removes a core tissue sample of 5 mm to 20 mm in diameter. While the ABBI has the advantage of providing a large tissue sample, similar to that obtained from an open surgical biopsy, the cylindrical tissue sample is taken from the subcutaneous tissue to an area beyond the suspect tumor. For tumors embedded more deeply within the breast, the amount of tissue removed is considerable. In addition, while less expensive than open surgical biopsy, the ABBI has proven expensive compared to other biopsy techniques, and it has been noted that the patient selection for the ABBI is limited by the size and location of the tumor, as well as by the presence of very dense parenchyma around the tumor. For discussion on the ABBI, see:
(3) Parker, Steve H. “The Advanced Breast Biopsy Instrumentation: Another Trojan Hourse?” Am. J. Radiology 1998; 171: 51-53.
(4) D'Angelo, Philip C., et al. “Stereotactic Excisional Breast Biopsies Utilizing the Advanced Breast Biopsy Instrumentation System.” Am J Surg. 1997; 174: 297-302.
(5) Ferzli, George S., et al. “Advanced Breast Biopsy Instrumentation: A Critique.” J Am Coll Surg 1997; 185: 145-151.

Another biopsy device has been referred to as the Mammotome and the Minimally Invasive Breast Biopsy (MIBB). These devices carry out a vacuum-assisted core biopsy wherein fragments of suspect tissue are removed with an 11 to 14 gauge needle. While being less invasive, the Mammotome and MIBB yields only a fragmentary specimen for pathological study. These devices therefore are consistent with other breast biopsy devices in that the degree of invasiveness of the procedure necessarily is counterbalanced against the need for obtaining a tissue sample whose size and margins are commensurate with pathology requirements for diagnosis and treatment.

In U.S. Pat. No. 6,277,083 B1 by Eggers, et al., issued Aug. 21, 2001, an instrument for removing a target tissue volume in a minimally invasive manner is described. That instrument includes a tubular delivery cannula of minimum outer diameter, for example, 6 mm, the tip of which is positioned in confronting adjacency with a tissue volume to be removed by extending it into a preliminary incision. Following such positioning, the electrosurgically excited leading edge of a capture component fashioned of a plurality of flexible leaf members combined with cutting and pursing cables are extended forwardly from the instrument tip to enlarge while electrosurgically cutting and surmounting the tissue volume, whereupon the cables are pursed to gather together the leaf tips and fully sever the targeted tissue from adjacent healthy tissue. Following such capture, the instrument and encaptured tissue volume are removed through the initial incision.

In a co-pending application for United-States-patent entitled “Minimally Invasive Intact Recovery of Tissue”, Ser. No. 09/904,396 by Eggers et al., filed Jul. 12, 2001, improvements are described in connection with the above-described capture component-based instrument with respect to both the configuration of the capture component and the structuring and methodology associated with the positioning of the tip of the instrument in confronting adjacency with targeted tissue. In the latter regard, electrosurgically excited precursor electrodes are located at the cannula tip and are so excited for the purpose of facilitating movement of the tip into the noted confronting adjacency with targeted tissue volume. Because electrosurgical cutting with these precursor electrodes involves the formation of a cutting arc it is necessary that the precursor electrodes be properly positioned subcutaneously before their energization. This calls for an initial cold scalpel incision through the skin layer which preferably is of a minimal dimension to avoid scaring and disfigurement. Such initial procedure, wherein the tip of the cannular instrument must be properly positioned before the precursor electrodes are electrosurgically excited, is important. For instance, the arc created by the precursor electrodes must be at a depth within subcutaneous tissue effective to avoid the creation of burns at the surface of the incision. While the skin creates an impressive barrier to externally asserted thermal attack, such a barrier effect is compromised when the thermal attack originates below the skin layer. Thus, enhancement of the initial steps of the process for target tissue recovery will be beneficial.

Another aspect of this target tissue accessing procedure is concerned with advancing the tip of the tissue recovery instrument from its subcutaneous starting position into confronting adjacency with the target tissue volume. The tissue encountered during this placement maneuver will vary. For instance adipose tissue typically will be encountered in the breast. Excessive tissue resistance to the instrument movement not only makes the procedure arduous but also may displace the target tissue volume to an extent defeating an incident-free guidance plan strategy. Forward tissue cutting by the precursor electrodes during this instrument positioning procedure must be adequate to permit device movement without substantial tissue resistance while avoiding excessive tissue damage.

### BRIEF SUMMARY OF THE INVENTION

The present invention is addressed to method and apparatus for accessing a target tissue volume with a tissue recovery instrument. With the method, upon determining the instrument entry location and attitude at the skin surface, a cold scalpel incision is made through the skin. That incision will have a length generally corresponding with the cross-sectional dimension of the cannular instrument tip and a depth effective to avoid thermal damage by electrosurgically excited precursor electrodes located forwardly of the instrument tip surface. To assure the proper initial positioning of these precursor electrodes prior to their electrical excitation, the incision is expanded with a pair of retractor components having mutually outwardly disposed tissue engagement surfaces dimensioned to establish a correct precursor electrode subcutaneous positioning depth at their tips. The retractor components are structured at their internal surfaces in correspondence with a cross-section of the recovery instrument so as to define an insertion entry mouth and a centrally disposed instrument guidance channel upon their actuation. Before the excitation of the precursor electrodes, the retractor components are removed from the incision by a sliding action along the surface of the tissue recovery instrument. This removal activity functions to assure a proper initial “setting” of the precursor electrodes by frictionally pulling the skin and tissue generally outwardly while the instrument remains in a stable position.

To facilitate the movement of the instrument forward portion towards a position of confronting adjacency with the target tissue volume, the precursor electrode assemblage is configured with radially disposed, thin flexible electrode branches which function having an equivalent diameter which is at least 90% of the corresponding cross-sectional diameter of the forward region of the recovery instrument. Preferably, four such precursor electrode branches are provided, arranged in symmetry or quadrature. Of additional importance, the coplanar forwardly disposed cutting surfaces of the precursor electrode branches are spaced forwardly from the surface of the tip of the recovery instrument. This provides an open region permitting forward displacement of the immediately cut tissue as the cannular instrument is moved toward its confronting orientation with the target tissue volume.

As another feature of the invention an insertion instrument is provided for aiding the positioning of a tip of a cannular instrument at a select depth within an incision of predetermined length extending along the skin of the patient, such tip carrying a forwardly disposed energizable cutting component. A first retractor component is provided which is moveable along a first retraction locus, the first retractor component having an outwardly disposed first tissue engagement surface extending along a first axis from a first tip, this first tip having a first insertion entry dimension. The first tissue engagement surface extends an insertion depth length corresponding with the incision select depth to a first insertion position. At that first insertion position, the tissue engagement surface exhibits a dimension generally transverse to the first axis having an extent generally corresponding with the cross-sectional dimension of the instrument surface and having an oppositely disposed generally concave first instrument guide surface at least a portion of which is contoured in correspondence with a first portion of the instrument surface in an amount effective to engage the instrument in generally guiding slidable relationship.

A second retractor component is provided which is moveable along a second retraction locus generally aligned with and oppositely directed from the first retraction locus. This second retractor component has an outwardly disposed second tissue engagement surface extending along a second axis from a second tip. This second tip has a second insertion entry dimension. The second tissue engagement surface extends an insertion depth length corresponding with the depth of the incision to a second insertion position. At that position, the second tissue engagement surface has a dimension generally transverse to the second axis of extent generally corresponding with the cross-sectional dimension of the surface of the instrument. The second retractor component has an oppositely disposed generally concave second instrument guide surface which is contoured in correspondence with a second portion of the instrument surface opposite the first portion an amount effective to engage the instrument in generally guiding slidable relationship. At least one of these first and second retractor components is movable along a corresponding locus toward and away from an initial orientation of mutual adjacency.

As another feature and object of the invention an electrosurgical instrument is provided comprising a support member having a central axis and surface of given radius, a circumference and diameter and having a forwardly disposed tip surface configured for movement through tissue toward a select location within the body of the patient. An energizable cutting electrode assembly is provided having at least three thin electrode branch portions, energizable to cut the tissue, which are arranged generally normally to and generally symmetrically about the central axis, the branch portions being located forwardly from the tip surface a spacing distance effective to enhance the forward displacement of the tissue when cut to facilitate a slidable engagement of the cut tissue with the support member surface. A source is provided which is actuable to apply cutting energy to the electrode branch portions.

Another feature of the invention is to provide a method for accessing a target tissue volume of predetermined diametric extent located beneath the skin of a patient with the tip of a cannular instrument having a given cross-sectional dimension and carrying a forwardly disposed energizable cutting assembly which comprises the steps of:

(a) determining an instrument entry location upon the skin;

(b) making an incision through the skin at the entry location having an incision length at least corresponding with the cross-sectional dimension of the instrument at an incision depth effective to avoid thermal damage to the dermis of the skin when the cutting assembly is energized;

(c) providing a retractor assembly having first and second retractor components each having an outwardly disposed engagement surface extending from a tip an insertion entry length corresponding with the incision depth to an insertion position and having mutually inwardly disposed generally concave and generally cylindrically-shaped instrument guide surfaces, and a retractor drive assembly actuable to move at least one of the first and second retractor components from an initial mutually abutting position to oppositely disposed spaced apart retracting positions;

(d) inserting the first and second retractor component tips within the incision to locate the incision position at the surface of the skin;

(e) actuating the retractor drive assembly to move at least one of the first and second retractor components to their spaced apart retracting positions wherein the instrument guide surfaces are mutually spaced apart a distance at least corresponding with the instrument given the cross-sectional dimension to define a guidance channel;

(f) inserting the instrument tip along the guidance channel to a position adjacent the first and second retractor component tips;

(g) removing the first and second retractor components from the incision;

(h) applying cutting energy to the cutting assembly; and

(i) positioning the instrument tip into confronting adjacency with the target tissue volume.

Other objects of the invention will, in part, be obvious and will, in part, appear hereinafter. The invention, accordingly, comprises the method and apparatus possessing the construction, combination of elements, arrangement of parts and steps which are exemplified in the following detailed description.

For a fuller understanding of the nature and objects of the invention, reference should be made to the following detailed description taken in connection with the accompanying drawings.

### BRIEF DESCRIPTION OF THE DRAWINGS

FIG. 1 is a perspective view of a section of human skin;

FIG. 2 is a top view of an insertion instrument employed with the invention;

FIG. 3 is a side view of the insertion instrument of FIG. 2;

FIG. 4 is a top view of the instrument of FIG. 2 showing it in an actuated orientation;

FIG. 5 is a sectional view of a forward portion of a retrieval instrument employed with the method of the invention;

FIG. 6 is a front view of the instrument shown in FIG. 5;

FIG. 7A-7C combine as labeled thereon to provide a flow chart describing the method of the invention;

FIG. 8 is a perspective view showing an initial step in the method of the invention;

FIG. 9 is an anatomical sectional view showing an initial incision carried out in conjunction with the method of the invention;

FIG. 10 is a perspective view showing a step in the method of the invention employing the instrument of FIG. 2;

FIG. 11 is an anatomical sectional view showing portions of the instrument of FIG. 2;

FIG. 12 is a perspective view showing another step in the method of the invention and revealing a tissue recovery instrument;

FIG. 13 is an anatomical sectional view showing the orientation of the instrument of FIG. 2 corresponding with FIG. 12;

FIG. 14 is a partial bottom view showing the orientation of the insertion and recovery instrument illustrating their joint use;

FIG. 15 is a sectional anatomical view showing the orientation of the insertion and recovery instrument in the course of carrying out the method of the invention;

FIG. 16 is a perspective view illustrating the method of the invention while employing a target tissue volume recovery instrument;

FIG. 17 is an anatomical sectional view illustrating instrument and anatomical orientations following the removal of an insertion instrument from an incision;

FIG. 18 is a front view of the instrument of FIG. 5 showing an alternate embodiment for a precursor electrode assembly;

FIG. 19 is a schematic representation of a two branch precursor electrode assembly;

FIG. 20 is a schematic representation of a three branch precursor electrode assembly;

FIG. 21 is a schematic representation of a four branch precursor electrode assembly;

FIG. 22 is a schematic representation of the instrument of FIG. 5 showing its tip in a position of confronting adjacency with a target tissue volume;

FIG. 23 is a schematic representation of the instrument shown in FIG. 22 subsequent to the deployment of a capture component;

FIG. 24 is a schematic representation of the instrument shown in FIG. 23 as it is being withdrawn from tissue;

FIG. 25 is a schematic representation of a retrieval instrument employing a precursor electrode assemblage having branches of larger radial extent for use in conjunction with a target tissue volume of larger extent;

FIG. 26 is a schematic representation of the instrument of FIG. 25 showing target tissue volume capture; and

FIG. 27 is a schematic representation of the instrument of FIG. 26 illustrating instrument and target tissue volume specimen withdraw.

### DETAILED DESCRIPTION OF THE INVENTION

As a prelude to considering the method and apparatus involved with the initial subcutaneous positioning of the then un-energized tip of the tissue capture instrument, some insight into the mechanical structure of tissue involvement may be beneficial. The initial tissue to be encountered in the procedure is the skin, which is an anatomically and physiologically specialized boundary lamina ranging from about 1.5 mm to 4.0 mm in total thickness. Structurally, skin is complex and highly specialized, being formed as an intimate association between two distinct tissues: keratinized stratified, squamous, epithelium, superficially, the epidermis, and a deeper layer of moderately dense connective tissue, the dermis. This combination results in an integument providing a most effective barrier against a variety of externally encountered phenomena including thermal and mechanical excursions.

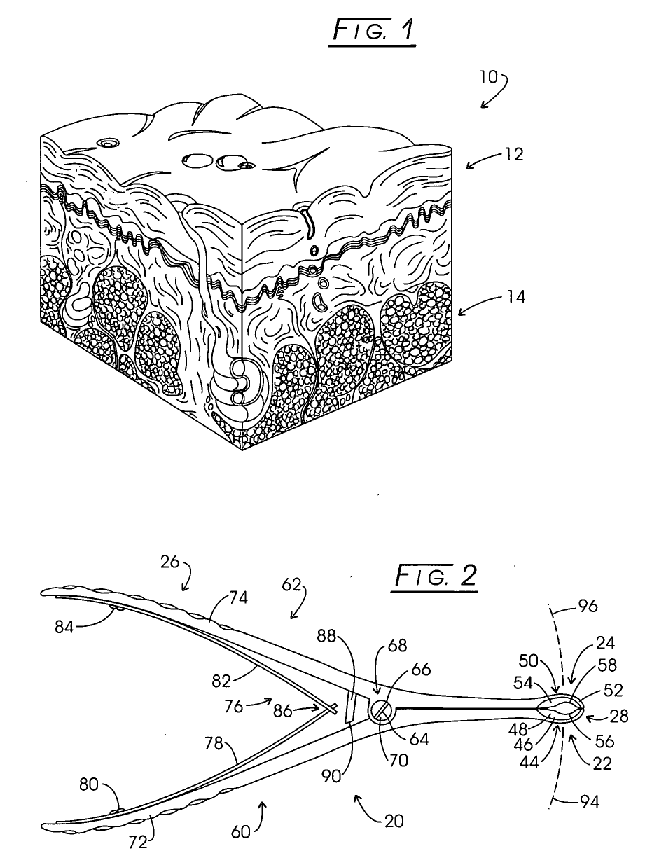

Referring to FIG. 1, a schematic representation of the organization of the skin is represented at 10. The somewhat thinner epidermis is represented generally at 12. In this stratified tissue 12 there is a continuous replacement of cells, a mitotic layer at the base replacing cells shed at the surface. As they move away from the base of the epidermis, they undergo progressive changes in shape and content, eventually transforming from polygonal living cells to dead, flattened squames full of the protein keratin. Below the epidermis 12 is the dermis, represented generally at 14. The dermis consists of irregular, moderately dense, soft connective tissue. Its matrix consists of an interwoven collagenous meshwork, with varying contents of elastin fibres, proteoglycans, fibronectin and other matrix components, bloodvessels, lymphatic vessels and nerves. Accordingly, it is of importance to avoid dermal damage to this skin structure arising from a heat source located subcutaneously, i.e., the barrier strata assembly normally imposed against externally induced thermal phenomena is reversed and thus jeopardized. In general, for the procedures of target tissue extraction, that tissue below the skin will be somewhat soft and palpable. This is particularly true in connection with the female breast which, in general, consists of glandular tissue and fibro-adipose tissue between its glandular lobes and lobules, together with blood and lymph vessels and nerves. See generally: Gray's Anatomy, P. L. Williams, et al, thirty-seventh Edition, Introduction, Splanchnology, Churchill Wadsworth Livingstone, N.Y., 1989.

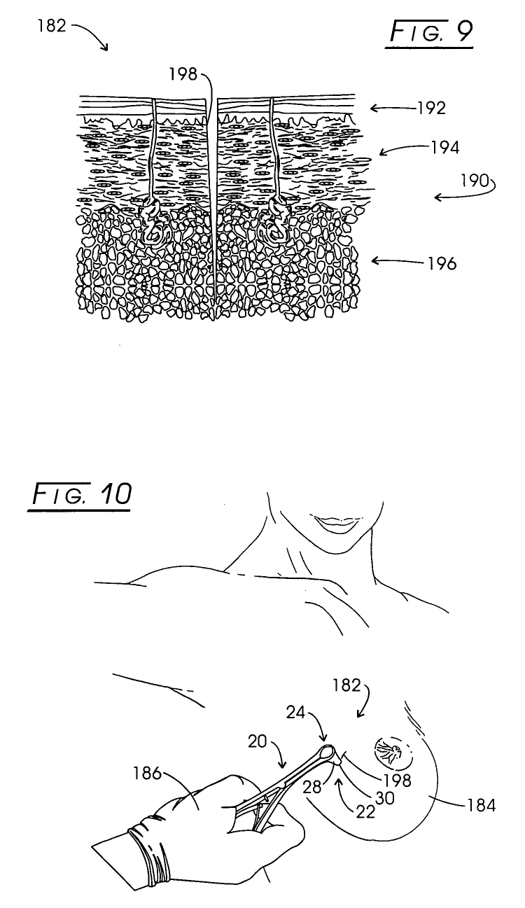

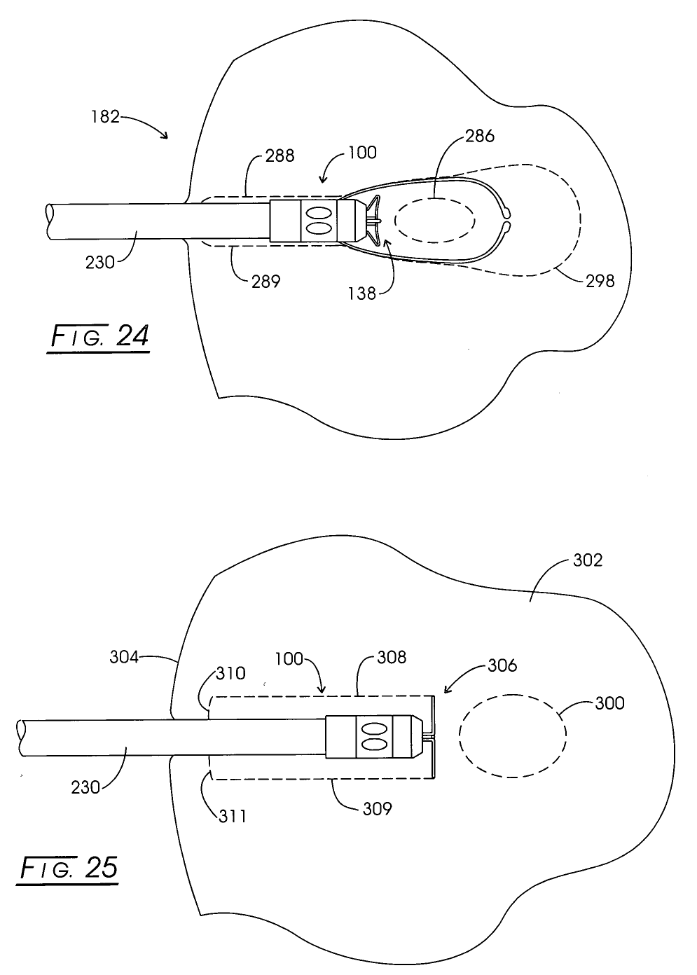

Retractor apparatus employed with the method of the invention is illustrated in connection with FIGS. 24. Referring initially to FIGS. 2 and 3, an insertion instrument is represented generally at 20. The instrument 20 is provided having two retractor components represented generally at 22 and 24 in FIG. 2. Component 22 is configured having an outwardly disposed convexly-shaped tissue engagement surface represented in general at 28 in FIGS. 2 and 3. FIG. 3 reveals that the tissue engagement surface 28 extends from a tip 30 to an insertion position represented at visible indicia configured as a groove 32 extending generally normally to the axis 34 of retractor component 22. A corresponding visible indicia 33 is provided in conjunction with a “mirror image” tissue engagement surface 29 extending to a tip 31 (FIGS. 9-10). The insertion position represented at indicia 32 is spaced from the tip 30 an insertion depth length represented by the arrows 38-38. Correspondingly, the tip 30 is configured having an insertion entry dimension represented by the arrows 38-38. Insertion position 32 is configured having an insertion position dimension represented at arrows 40-40, again taken generally normally to the axis 34. FIG. 3 additionally reveals that the retractor component 22 includes an upwardly disposed threshold portion 44. As shown in FIG. 2, the threshold portion 44 is outwardly tapering to define the entrance mouth portion of a generally concave instrument guide surface 48. Guide surface 48 is contoured in correspondence with a forward portion of the surface of the capturing recovery instrument which, for the present embodiment is a cylindrical surface.

Retractor component 24 is similarly configured, having a threshold portion represented generally at 50, forming an entrance mouth portion 52 which, in turn, is integrally formed with and configured as an extension of an instrument guide surface 54. Guide surface 54 is generally concavely contoured in correspondence with the tip region cylindrical surface of the associated capture instrument. Retractor components 22 and 24 are shown in FIG. 2 in an initial orientation of the instrument guide surfaces respectively described at 46 and 54. Note that the contour of these surfaces when combined together at this initial orientation, appears somewhat ovuloid. Accordingly, it may be observed that these instrument guide surfaces have a profile at the tips 30 and 31 which represents a portion of a circle as seen, respectively at guide surface tip profiles 56 and 58. Profiles 56 and 58, when engaged with the cylindrical capturing recovery instrument provide a contour which corresponds with it. For the capturing recovery instrument described above, a diametric extent of about 6 mm is provided in the region of its tip. The figure additionally reveals that the tissue engagement surfaces and associated oppositely disposed guide surfaces mutually taper inwardly toward the tip profiles 56 and 58 to facilitate their insertion within an incision.

FIG. 2 further reveals that the retractor drive assembly 26 includes a lever represented generally at 60 which is integrally coupled with retractor component 22 and a lever represented generally at 62 which is integrally coupled with retractor component 24. Each of the levers 60 and 62 extend from their respective retractor components 22 and 24 a pivot distance to respective pivot locations 64 and 66. The levers are mutually pivotally connected at a connector assembly represented generally at 68 comprised of a hinge formation including a machine screw 70. Lever 60 extends from the pivot location 64 to define a manually graspable handle component 72. Correspondingly, lever 62 extends from the pivot location 66 to define a manually graspable handle component 74. Handle components 72 and 74 are mutually biased outwardly by a spring assembly represented generally at 76. Assembly 76 includes a spring leaf 78 connected by machine screw 80 to handle portion 72 and a spring leaf 82 connected to handle component 74 by a machine screw 84. Spring leafs 78 and 82 are slidably joined together with a tongue and groove assembly 86. Note, additionally, that a stop member 88 is fixed to and extends outwardly from the inner surface of handle component 74 toward the corresponding internal surface of handle component 72. The optional stop member is shown having a tip or abutting surface 90 and functions to limit the extent of the spacing of retractor component 22 from retractor component 24. This limitation functions to prevent tearing of tissue with the instrument 20. The stop member can be made adjustable, for example, by providing a threaded connection with handle component 74.

Returning to FIG. 3, tissue engagement surface 28 is seen to be optionally configured with discontinuities represented generally at 92 and here implemented as a plurality of grooves arranged generally normally to the retractor component axis 34. The corresponding tissue engagement surface 29 of retractor component 24 is similarly configured with discontinuities 93 (FIGS. 90-91). This discontinuity of the surfaces as represented at 92 functions in particular when those surfaces are removed from an incision in preparation for the energization of a precursor electrode assembly. The discontinuities tend to engage skin and subcutaneous tissue while the recovery instrument is held steady, i.e. stabilized to assure proper depth positioning of the precursor electrode.

Referring to FIG. 4, instrument 20 is shown in an orientation where it has been manually actuated to the extent wherein the abutting surface 90 of stop member 88 has engaged the internal surface of handle component 72. This will have caused one or both of retraction components 22 and 24 to move along respective retraction locii shown at 94 and 96 to establish a distance between guide surface tip profiles 56 and 58 effective to receive the noted cannular forward portion of the capturing recovery instrument.

The recovery instrument with which the insertion instrument 20 is utilized may generally be categorized as incorporating an elongate delivery cannula of quite small diametric extent which extends to a forward region and tip. Extending from he forward surface of the tip is an electrosurgically excitable thin, flexible, wire-form precursor electrode configuration. Just rearwardly of the tip, the delivery cannula encloses the rearward components of capture component elongate but diminutive stainless steel leafs each forwardly terminating in an eyelet structure through which electrosurgically excitable pursing and cutting cables extend. It is this tip region with which the guide surfaces 48 and 54 as well as tips 30 and 31 become operationally associated.

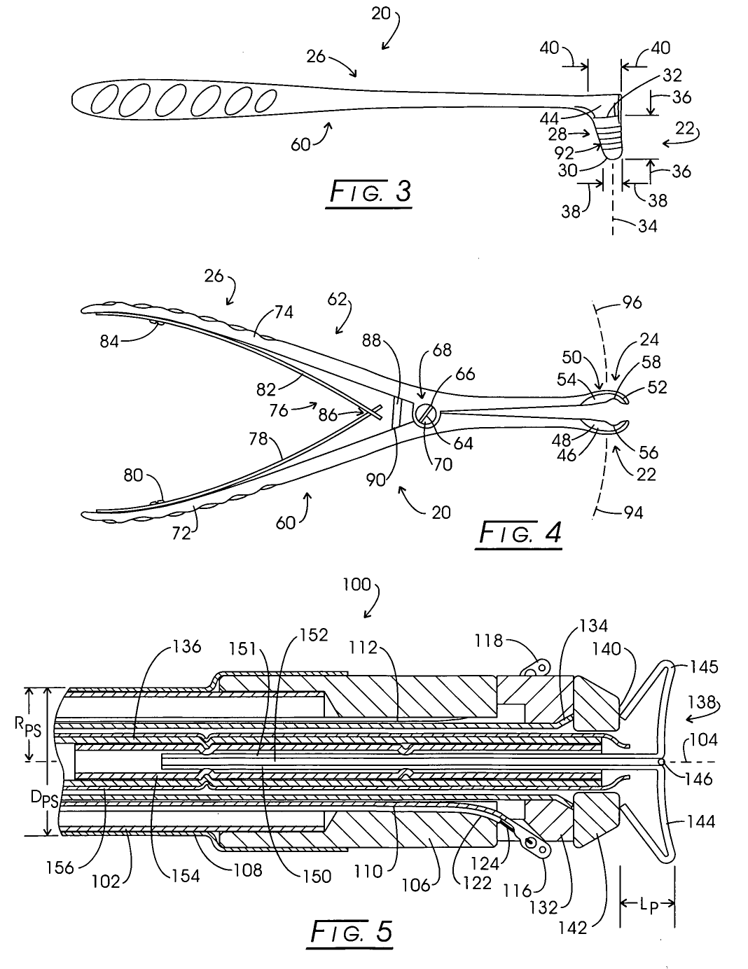

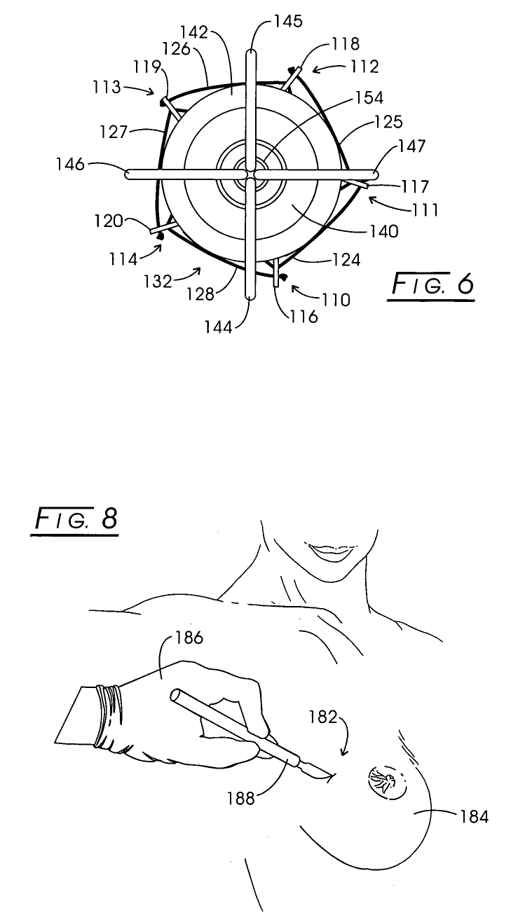

Referring to FIG. 5, this tip region is represented generally at 100 in sectional fashion. The figure reveals the forward portion of a cylindrical or tubular stainless steel delivery cannula 102 which is symmetrically disposed about an axis 104. Delivery cannula tube 102 extends forwardly to support a cylindrical polymeric (e.g., polyetherimide) rearward tip component 106. Delivery cannula 102 is electrically insulated with a five mil thick polyolefin shrink tube 108 which additionally extends over the cylindrical outer surface of rearward tip component 106. Next inboard from the internal surface of the delivery cannula 102 are five capture component leafs arranged in a pentagonal configuration, two of which are seen at 110 and 112. Each of these five leafs are identically structured. Note that leaf 110 is seen to support a thin polyamide cable guide tube 122 extending longitudinally along the center of its outside surface. A very thin braided, electrically conductive pursing and cutting cable 124 is seen extending from the guide tube 122 into a pursing association with the eyelet structure 116 of leaf 110.

Not seen in the rearward tip component 106 are a plurality of smoke/steam/fluid evacuation ports which communicate in vacuum association with an evacuation channel established initially as a gap between the outer surface of the leafs as at 110 and 112 and the internal surface of rearward tip component 106. The channel then extends rearwardly as a gap adjacent to the internal surface of delivery cannula 102 to a suction deriving assembly (not shown). An evacuation of accumulations of fluid such as local anesthetic and blood is important for assurance of an electrode-derived tissue cutting arc.

Extending next inwardly inboard is an elongate stainless steel support tube 130 which is seen to extend through rearward tip 106 and into engagement with a forward tip component 132. This engagement is improved by a flairing at the forward end 134 of tube 130. Located inside the support tube 130 is a precursor electrode tube 136 which supports a precursor electrode assembly represented generally at 138. Assembly 138, for the instant embodiment, comprises for precursor electrodes which extend forwardly of the forward surface 140 of a ceramic tip 142 attached, in turn, to forward tip component 132. Three of the four somewhat flexible stainless steel-precursor electrode wires are shown at 144-146. Each of the four stainless steel precursor electrode wires is configured with a generally elongate L-shape, including an elongate shank region or shaft, three of which are shown at 150-152 in conjunction with respective electrodes 144-146. These four electrode shanks or shank regions are crimped inside of a tube 154 and that tube 154, in turn, is crimped within the forward portion of the precursor electrode tube 136. Electrosurgical energy is delivered to the precursor electrodes via this tubular configuration. Accordingly, precursor electrode tube 136 is insulated with an electrically insulating shrink wrap 156.

Referring additionally to FIG. 6, a preferred arrangement of four precursor electrodes is revealed at 144-147, the electrodes being arranged in quadrature or symmetrically about the instrument axis 104. In general, these precursor electrodes 144-147 will have a tissue cutting and confronting diametric length of about 6.5 to 7.0 mm, while the tip region 100 of the instrument will exhibit a diametric extent of about 5.5 mm. The noted precursor dimension is selected where the target tissue volume exhibits a general diametric extent of up to about 15 to about 20 mm. As the target tissue volume diametric extent expands therefrom then the precursor electrode diametric extent expands accordingly. For all applications, the electrode wires are of a thickness permitting their flexure. In this regard, they will exhibit a diameter of from about 0.05 mm to about 0.5 mm.

FIG. 5 reveals that the severing portions or tissue confronting portions of the precursor electrodes extend generally normally to the longitudinal instrument axis 104. Of importance, the figure shows that these confronting surfaces are spaced a distance Lₚ from the ceramic cap surface 140.

Now considering the capture procedure, the rearward tip component 106 functions as a confinement or alignment sleeve for each of the five leafs of the capturing assembly. In this regard, the component functions in conjunction with tip portion 132 to establish five pentagonally oriented ramps to provide initial guidance for the leafs as they are emerging during the capture procedure.

Returning to FIG. 6, the pentagonally associated eyelet structures 116-120 for respective leafs 110-114 are revealed. As the leafs emerge from the ramp structures, the pursing and cutting cables shown in FIG. 6 at 124-128 are initially played out and then are tensioned to define both the size and the shape of the capture cut and implementing cage-like capture component developed with the procedure.

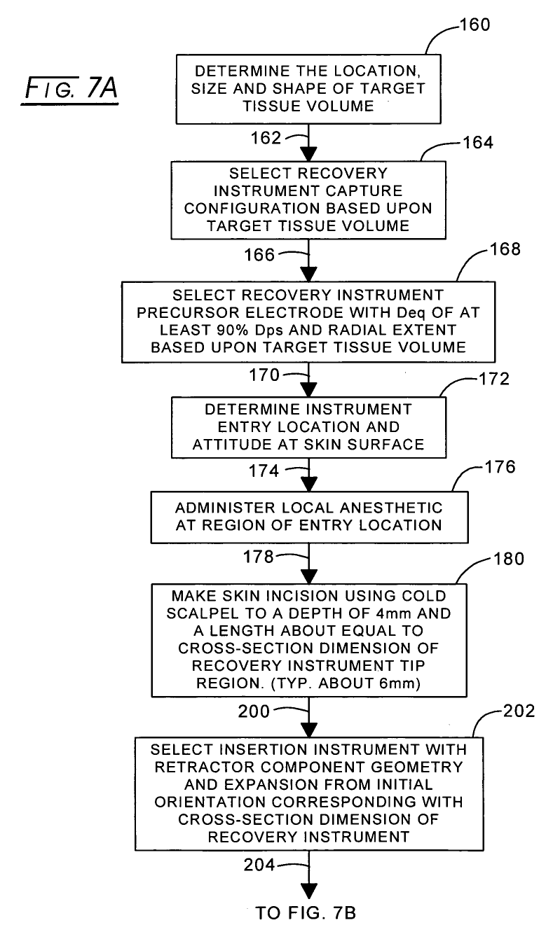

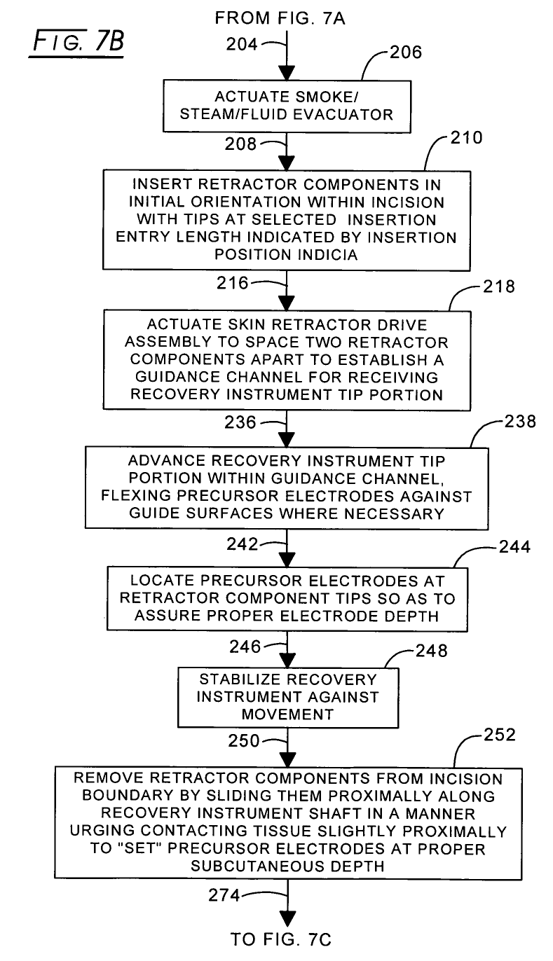

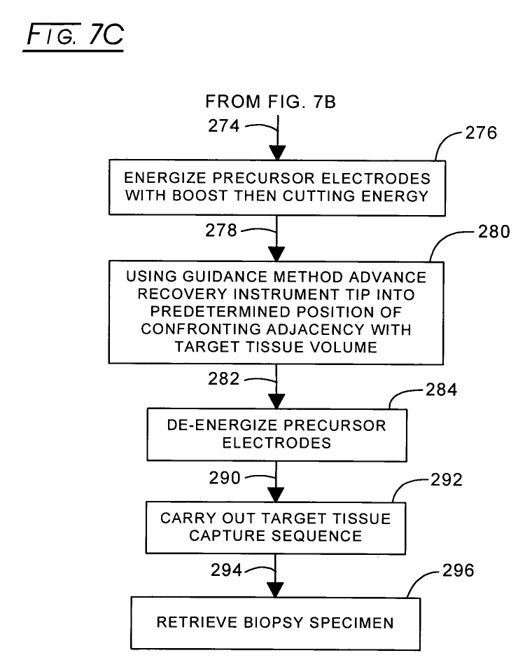

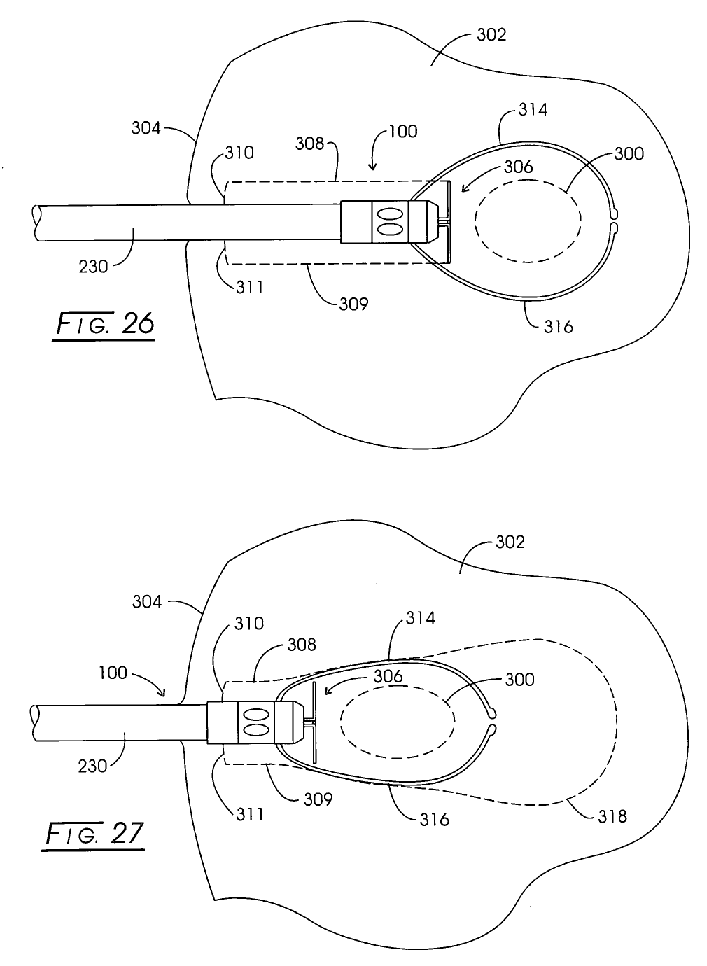

The method for accessing a target tissue volume in accordance with the invention in its early stages involves the utilization of the retractor instrument 20 and then is concerned with the configuration of the precursor electrodes at the recovery instrument tip as it is devised to substantially facilitate the movement of the instrument tip into a predetermined confronting adjacency with the target tissue volume. Accordingly the discourse to follow looks initially to the manipulation of the retractor instrument 20 and the initial positioning of the recovery instrument as well as its precursor electrode assembly and selection. The entire accessing procedure is outlined in conjunction with the flow chart represented at FIG. 7A-7C; is illustrated in connection with FIGS. 8, 10, 12 and 16; and is anatomically described in connection with the FIGS. 9, 11, 13, 14, 15 and 17. Precursor electrode structuring and theory is discussed in connection with FIGS. 5, 6, 18 and 19-21. Finally, a brief discourse is provided concerning the removal of a captured target tissue volume in connection with FIGS. 22-27.

Looking to the procedural chart commencing with FIG. 7A, opening block 160 provides for a determination of the location, size and shape of the target tissue volume. This typically is carried out utilizing any of a variety of diagnostic tools and procedures which historically have shown continuous improvement and which continue to improve. It is that size and shape of the target tissue volume which contributes the data necessary for electing the capturing configuration and precursor electrode effective diametric extent for reaching a confronting attitude with respect to the tissue volume with the recovery instrument tip. As represented at arrow 162 and block 164, the procedure selects a recovery instrument capture configuration based upon the target tissue volume size and shape. The particular recovery instrument achieves those variations by select tensioning of the pursing and cutting cables described in connection with FIG. 6 at 124-128. As represented at arrow 166 and block 168, a part of the recovery instrument configuration accessing the size of the target tissue volume involves the concomitant selection of the precursor electrode configuration. As the tip of the instrument reaches a position of predetermined confronting adjacency with the target tissue volume, it is necessary that such movement of the instrument through tissue be carried out both with unnecessary destruction to the involved tissue and also with such facility that the movement of the instrument is relatively unrestrained, particularly to the extent that the position of the target volume is not altered in consequence of deformed adjacent tissue regions, such deformation being occasioned by instrument movement. Note that block 168 calls for the selection of the recovery instrument precursor electrode with an effective diameter, D_{ez} of at least 90% of the diameter of the cannular instrument component itself, Dₚₛ. The radial or diametric extent of the precursor electrode is elected based upon target tissue volume size. With the above determinations being made, as represented at arrow 170 and block 172 the practitioner determines the instrument entry location as well as its attitude with respect to the skin surface about to be invaded. In this regard, the elongate cannular component of the recovery instrument is in effect, “aimed” so as to position its tip at the noted confronting orientation. Instrument movement in this regard can be manually evolved or developed through the utilization of steriotactic devices. Once the determination represented at block 172 is made, then as represented at arrow 174 and block 176 a local anesthetic is administered at the now determined region of entry location for the recovery instrument. An initial skin incision now is made as represented at arrow 178 and block 180. This incision is made with a cold scalpel to a depth of about 4 mm for recovery instruments with precursor electrodes configured and sized for the recovery of, for example target tissue volumes in the range of about 10 mm to about 15 mm. The length of this incision is made about equal to the cross-sectional dimension of the recovery instrument tip region which, for the disclosed embodiment will be about 6 mm.

Looking to FIG. 8, the general region of the noted entry location is shown generally at 182 in connection with a right breast 184. The practitioner's hand 186 is shown in the process of making this initial incision with a cold scalpel 188. Looking to FIG. 9, the region 182 is reproduced, an anatomical section of the skin being represented in general at 190 as including the epidermis represented in general at 192 and the dermis as represented at 194. Below the dermis 194 the female breast will exhibit relatively soft adipose tissue as represented at 196. Cold scalpel 180 will have created the noted incision as represented at 198, for the instant demonstration, to a depth of about 4 mm. This depth is elected such that when the forwardly disposed precursor electrode assembly 138 (FIG. 5) is initially positioned and then energized, when operated in concert with a steam/smoke/fluid evacuation feature, thermal damage to the skin region 190 will be avoided. Recall that the barrier to thermal phenomena normally established by the skin 190 is provided in a reverse laminar sense, i.e., protection normally is provided from the epidermis 192 with respect to external thermal phenomena.

Returning to FIG. 7A, following the formation of the incision 198, the procedure continues as represented at arrow 200 extending to block 202 wherein the procedure selects the insertion instrument having a retractor component geometry and expansion from an initial orientation corresponding with the cross-sectional dimension of the recovery instrument annular support. For the recovery instrument described in connection with FIG. 5, that cross-section remains consistent for essentially all of the tumor sizes encountered with the instant procedure. Accordingly, for the preferred embodiment, the stop member 88 described in conjunction with FIG. 2 may be of fixed length as illustrated in that figure.

The procedure continues as represented at arrow 204 and block 206 (FIG. 7B). The latter block provides for the actuation of a smoke/steam/fluid evacuation system portions of which have been described in conjunction with FIG. 5. This system provides suction at the instrument tip region 100. The procedure continues as represented at arrow 208 and block 210 providing for the insertion of the retractor components 22, 24 when in their mutually abutting initial orientation. Commencement of this insertion is shown in FIG. 10, the mutually abutting tips 30 and 31 extending into the incision 198 the selected insertion entry length, for example, about 4 mm. Looking additionally to FIG. 11, retractor components 22 and 24 are represented in conjunction with the anatomical features shown earlier in connection with FIG. 9. In the figure, the tips 30 and 31 are seen at the elected depth for the location of the precursor electrode assembly 138 (FIG. 5). The discontinuities represented in general at 92 and 93 within respective initial engagement surfaces 28 and 29 are herein implemented as a sequence of parallel grooves an uppermost pair of grooves as mutually aligned normally axis 34 are shown at 32 and 33 in adjacency with the outer surface of the epidermis 192. This assures an appropriate positioning of tips 30 and 31. Note, additionally, that this procedure of insertion will have caused an inward flexure of at least the epidermis 192 of skin 190 as depicted in FIG. 11 at 212 and 213. The overall effect of this initial insertion of the retractor components 22 and 24 may stress and thus strain adjacent tissue an extent potentially affecting requisite precursor electrode positioning subcutaneously. The groove-implemented discontinuities 92 and 93 at this juncture in the procedure will have some engagement with abutting tissue.

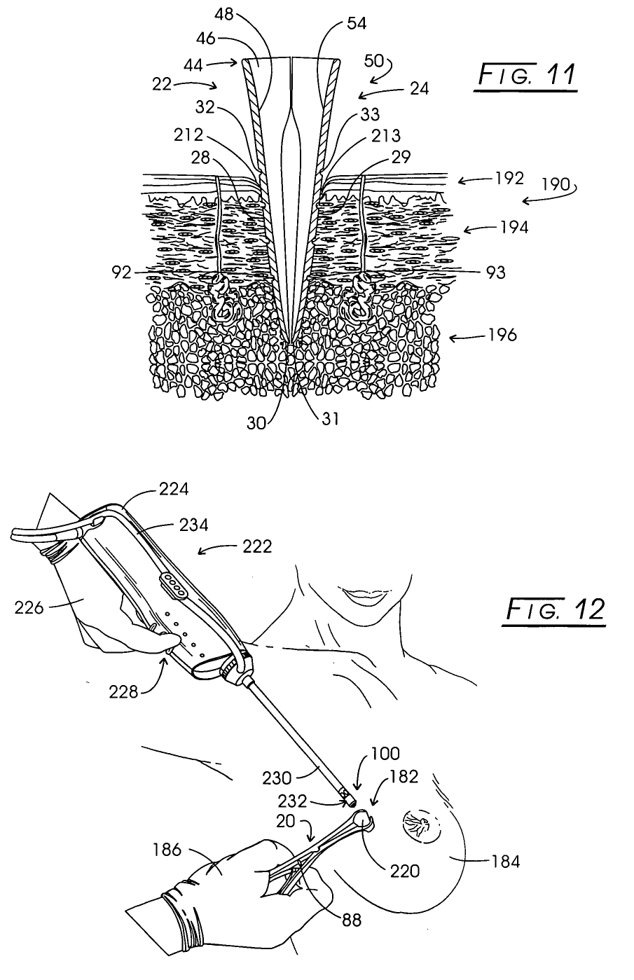

Returning to FIG. 7B, the procedure continues as represented at arrow 216 and block 218 providing for the actuation of the skin retractor drive assembly 26 to cause the relative spacing apart of the retractor components 22 and 24 to establish a guidance channel for receiving the recovery instrument tip portion 100. Looking to FIG. 12, the insertion instrument 20 is shown in this orientation wherein a guidance channel 220 has been established. The tip 100 of a recovery instrument represented generally at 222 is shown poised for insertion through the guidance channel 220 established by the insertion instrument 20.

Described in detail in the above-referenced application for U.S. patent Ser. No. 09/904,396, the instrument 222 is shown having a housing 224 being held and manipulated by the hand 226 of a practitioner, the device being hand-controlled by an array of button switches represented generally at 228. The tip 100 as described in conjunction with FIG. 5 is seen to be at the forward region of an elongate delivery cannula 230 extending, in turn, from housing 224. Suction ports represented generally at 232 are in communication with a suction system communicating with the ports 232 via the delivery cannula 230 and flexible suction tubing 234. While the instrument 222 can be sterotactically maneuvered, it also can be hand maneuvered as shown. This follows by virtue of the insertion technique and in consequence of a unique configuration of the precursor electrodes as described, for example, at 138 in FIG. 5. Note that no clamps or stabilizing implements are employed in conjunction with the breast 184. Of course, such clamps may be used, depending upon the desires of the practitioner. It is because of the ease of maneuvering the tip 100 into confronting adjacency with the target tissue volume that body region stabilization devices need not be used and the procedure may be carried out in a medical office environment.

Looking to FIG. 13, an anatomical representation of this step in the procedure is represented. Here the retractor components 22 and 24 are seen to have expanded the incision to establish an expanded incisional periphery. The tips 30 and 31 of respective retractor components 22 and 24 are at the appropriate insertion entry length from the epidermis 192 surface as verified by the location of the indicia 32 and 33 in adjacency therewith. Guidance channel 220 now is established for reception of the tip 100 of instrument 222.

Returning to FIG. 7B, as represented at arrow 236 and block 238, the recovery instrument 222 now is advanced to an extent that the tip portion 100 is extended within the guidance channel 220 of insertion instrument 20. Where necessary, the precursor electrodes will be flexed inwardly by virtue of their contact with the instrument guide surfaces 48 and 54.

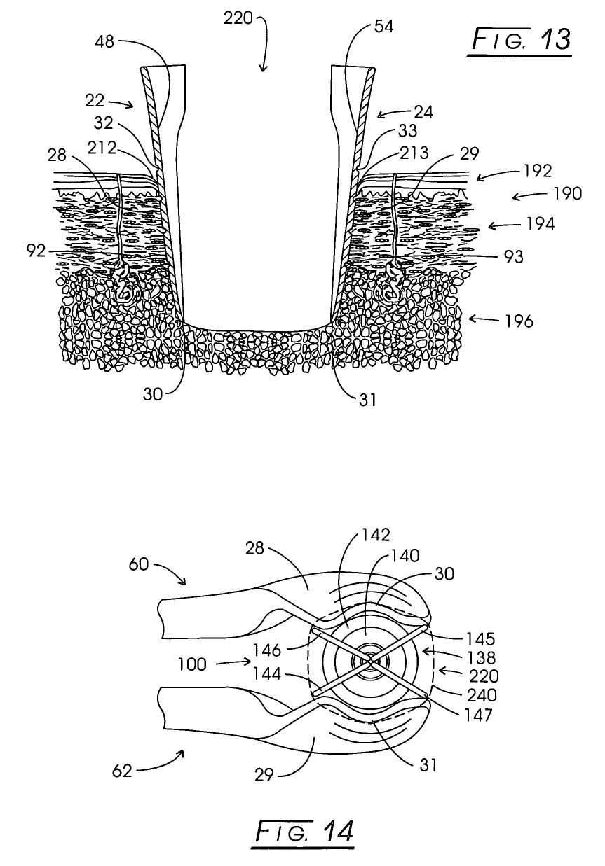

Referring to FIG. 14 tissue engagement surfaces 28 and 29 are seen to have enlarged the periphery of the incision as represented by the dashed incision enlargement boundary 240. Note, however, that the forwardly posed tissue confronting components 144-147 of precursor electrode assembly 138 have been flexed inwardly by the instrument guide surfaces 48 and 54 as they are advanced to adjacency with the tip peripheries 30 and 31. Thus, the precursor electrode assembly 138 has passed through boundary 240 at the epidermis 192 and is now located for resilient resumption of its initial symmetrical quadrature configuration within tissue 196.

Returning to FIG. 7B, the procedure then continues as represented at arrow 242 extending to block 244. Block 244 provides for the locating of the precursor electrode assembly at the retractor component 20 tips 30 and 31. This assures that the forward, tissue confronting and cutting surfaces of the electrodes as at 144-147 are properly located below the surface of epidermis 192 to avoid thermal damage emanating from those electrodes when they become excited. Looking to FIG. 15, electrode assembly 138 is seen thus located in adjacency with the tip peripheries 30 and 31. Note additionally that the retractor components 22 and 24 remain in place and that the surface of at least the epidermis remains flexed inwardly as represented at 212 and 213. The discontinuities 92 and 93, manifested as arrays of grooves, will have been compressibly engaged with surrounding tissue as at the dermal layer 194 and tissue 196.

Returning to FIG. 7B, an arrow 246 is seen extending from block 238 to block 248 providing for the stabilization of the recovery instrument against movement. This step is preliminary to and associated with the removal by the practitioner of the insertion instrument 20. With the instrument 222 stabilized, then as represented at arrow 250 and block 252 the practitioner removes the retractor components 22 and 24 from the incision boundary 240 (FIG. 14) by sliding them proximally along the recovery instrument delivery cannula or shaft. As this occurs, the discontinuities 90 and 91 (FIGS. 11, 13 and 15) will urge tissue adjacent the two engagement surfaces 28 and 29 outwardly towards the surface of the epidermis 192. As this is occurring, the instrument tip region 100 remains stationary or stabilized and the result is a “setting” of the precursor electrode assembly 138 at a proper subcutaneous depth.

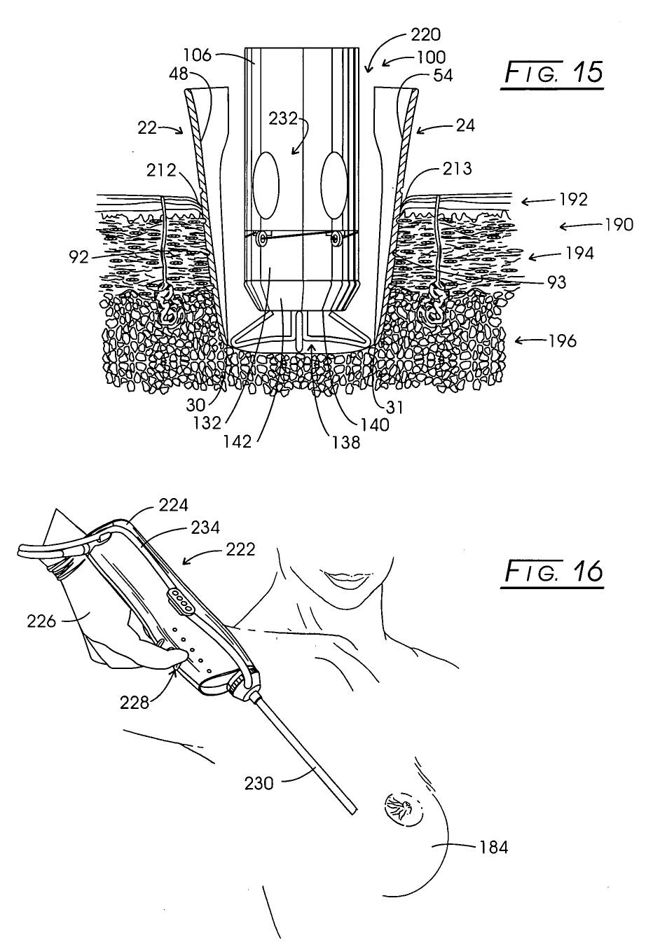

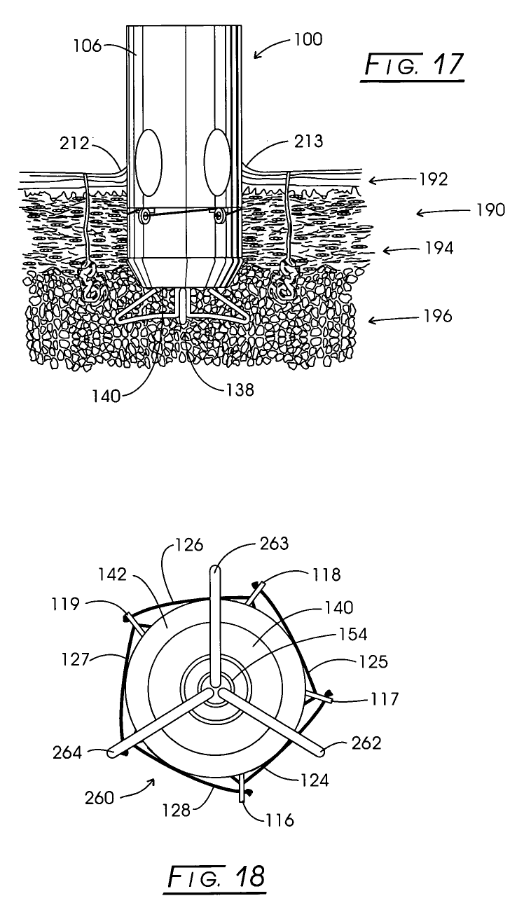

Referring to FIG. 16, the orientation of the recovery instrument 222 and its associated delivery cannula 230 is pictorially represented. Referring to FIG. 17 an anatomical representation of the status of the procedure is represented. Note that the tissue engaging and cutting surfaces of the precursor electrode assembly 138 remain in a stable position within tissue 196 at an appropriate depth beneath the surface of the epidermis 192. Because the retractor instrument has been removed by slidably proximately moving it along the forward surface region 100 of instrument 222, the discontinuance regions or grooves 92 and 93 have caused a somewhat outwardly oriented disposition of the flesh and skin. Note in this regard, that the epidermis regions 212 and 213 now are depicted as being outwardly flexed. It is at this juncture, that the electrode assemblage 138 is energized in preparation for maneuvering tip region 100 into confronting adjacency with the target tissue volume.

The discourse now turns to the configuration of the precursor electrode assembly with respect to the tip region of the instrument 222 delivery cannula which permits next maneuver to be carried out quite facily and with a minimization of damage to the tissue through which the tip region 100 courses.

As an initial consideration of the development of this facile cutting-based maneuver, reference again is made to FIG. 5 and, in particular, to the forwardly disposed location along central axis 104 of the tissue confronting surfaces of the four branches 144-147 of the precursor electrode assembly 138. Note in the figure that these branches and, in particular, their forward surfaces are spaced a spacing distance, L_{P} from the forward surface 140 of the delivery cannula. This spacing distance, L_{P} is selected as being effective to enhance the parting displacement of tissue when cut. This facilitates the slidable engagement of the outer surface of the delivery cannula with adjacent tissue. The spacing distance, Lₚ, will fall within a range of from about 0.5 mm to about 5.0 mm and preferably within a range of about 1 mm to about 2 mm.

A next consideration in facilitating this movement of the instrument to confronting adjacency resides in the geometric structuring of the precursor electrode assembly. Referring to FIG. 19, a precursor electrode assemblage, represented generally at 260, is shown having three generally L-shaped electrode branches 262-264 which are symmetrically disposed about the instrument axis and, as before, are retained by crimping within earlier-described tube 154. Symmetry is provided by disposing the cutting and confronting surfaces of the electrodes 262-264 at 120° intervals about the instrument axis.

The radial structuring of the precursor electrode branch confronting surfaces as at 138 and 260 is provided to achieve an effective or equivalent diameter of the opening which they create as the instrument passes through tissue. The larger this equivalent or effective diameter, the more facile will the instrument be maneuverable. However, as the equivalent diameter continues to increase, the result will be an unwanted degree of tissue injury which ultimately would approach the injury occasioned by creating a core, for example, with the advanced breast biopsy instrumentation.

An analysis of the noted equivalent diameter is presented in conjunction with FIGS. 5 and 19-21. In this regard, geometric notations are provided in conjunction with FIG. 5. In FIGS. 19-21, the circumference, Cₚₛ, of the forward region 100 of recovery instrument 222 is represented by circular dashed line 268. Accordingly, the boundary 268 necessarily must engage tissue as the instrument forward region 100 is maneuvered toward the target tissue volume. Each branch of the precursor electrode assembly is shown extending from the instrument central axis outwardly toward the boundary 268. FIG. 19 schematically portrays a two branch implementation of the precursor electrode assembly, the two electrode branches being represented at 270 and 272. Each of these branches is designated as extending along a radius of the boundary 268. Again referring to the geometric notations of FIG. 5, this provides for the following expressions:

Dₚₛ=2Rₚₛ (1)

Cₚₛ=2π×Rₚₛ=6.28Rₚₛ (2)

When the electrode branches 270 and 272 are electrosurgically excited, four radially identified cuts will be made in the tissue as the instrument is directed forwardly. Those cuts are numbered 14 in FIG. 19. The analysis now determines what equivalent diameter, Deq will be produced by the electrosurgical excitation of these two radially disposed electrode branches 270 and 272. To develop the value for Deq, it is assumed that the instrument will push forwardly, i.e., displace the cut tissue and that the extent of cutting with two branch electrodes 270 and 272 will evolve an effective “hole” size which may be identified by the noted equivalent diameter. The available circumferential extent for the two branch orientation is assigned as the sum of the cut surfaces 1-4 created by electrode branches 270 and 272. Accordingly, for this two branch version, the available equivalent circumferential extent, C_{2branch} will be represented as follows:

C_{2branch}=4Rₚₛ=π×D_{eq} (3)

²D_{eq}=C/π=4Rₚₛ/π=1.27Rₚₛ (where ²D_{eq} represents the equivalent diameter of incision produced by a two branch implementation of the precursor electrode assembly) (4)

Accordingly, for the two branch precursor electrode orientation shown in FIG. 19, the equivalent diameter is as follows:

²D_{eq}=63% Dₚₛ (5)

The above evaluation, 63% D_{PS}, has been found to be of value which is too low for achieving a desired facility of maneuvering of the instrument 222.

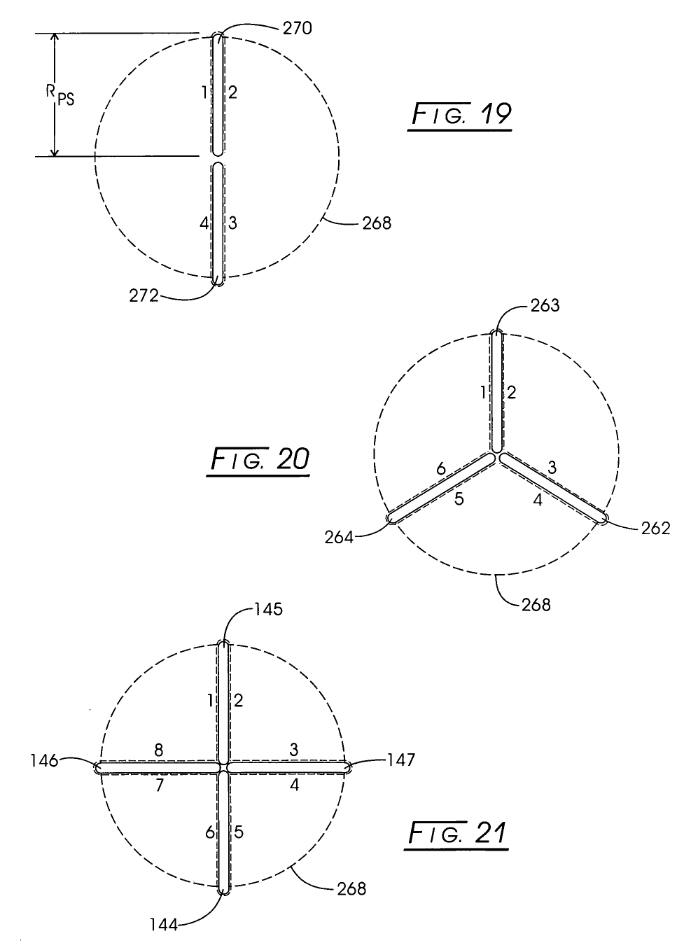

Looking to FIG. 20, the three precursor electrode branch configuration described in connection with FIG. 18 above is schematically portrayed in conjunction with the instrument forward region circumferential profile 268. Accordingly, the same numerical identification of the electrode branches is provided. For this three branch embodiment, it may be observed that six, cut tissue surfaces will be created as are numbered in the figure. Each of these cut tissue surfaces will be of a radial extent, Rₚₛ. Accordingly, as the forward region 100 of the instrument 222 is urged forwardly through the cut tissue surfaces and they are displaced, the available circumference is as follows:

C_{3branch}=6Rₚₛ Applying the above analysis the following relationships obtain (where ⁿD_{eq} refers to the equivalent diameter of incision produced by an n-branch precursor electrode assembly): (6)

³D_{eq}=C/π=6Rₚₛ/π=1.91Rₚₛ (7)

³D_{eq}=95.5% Dₚₛ (8)

Accordingly, by adding another radial branch to the precursor assembly, the effective cut diameter becomes 95.5% of the diameter of the forward portion 100 of the recovery instrument. This provides for an acceptable advancement maneuver of the instrument toward confronting adjacency with the target tissue volume.

FIG. 21 provides a schematic representation of the preferred, four branch precursor electrode assembly 138 described in conjunction with FIG. 6. Accordingly, the same numerical identification is provided for the precursor electrode branches in the instant figure. Looking to the figure, it may be seen that the radially disposed precursor electrodes 144-147 create eight cut tissue surfaces which are numbered 1-8 in the figure. As these cut surfaces are displaced forwardly-outwardly by the tip region 100 of the instrument 222 they may evoke an equivalent conferential extent of cut tissue represented as follows:

C_{4branch}=8Rₚₛ (9)

Applying the above analysis, the following relationship is obtain:

⁴D_{eq}=C/π=8Rₚₛ/π=2.55 Rₚₛ=127% Dₚₛ (10)

The above analysis, showing that the four branch embodiment for the precursor electrode achieves an equivalent diameter representing 127% of the diameter of the forward region 100 of instrument 122, confirms an experimentally established ease of maneuverability of the instrument tip region.

Returning to FIG. 7C, as represented at arrow 274 and block 276, the precursor electrodes are energized. For electrosurgical cutting requiring formation of an arc, this energization will include a short interval of energization at a boost voltage effective to commence formation of an arc. Following the boost voltage, the voltage level will drop to a cutting level. It is during this initial energization that the requisite subcutaneous positioning of the precursor electrodes avoids burn damage extending excessively back toward the skin surface. With the energization of the precursor electrodes, as represented at arrow 278 and block 280, using a guidance imaging method, for example, ultrasound or the like, the practitioner advances the recovery instrument 222 tip into a predetermined position of confronting adjacency with the target tissue volume. Then, as represented at arrow 282 and block 284 the precursor electrodes are de-energized. Looking momentarily to FIG. 22, a schematic representation of this procedure is portrayed. In the figure, a commonly encountered relatively smaller target tissue volume, for example, having a confronting diametric extent of about 10 mm is represented in dashed fashion at 286. Also shown in the figure in dashed line fashion at 288 and 289 is a representation of the outboard extent of the cut made by the precursor electrodes 138.

Returning to FIG. 7B, and looking to FIG. 7C as represented at arrow 290 and block 292, a recovery instrument is utilized to carry out a target tissue capture sequence. Following the cutting around and “caging” of the target tissue volume including healthy surrounding tissue the sequence provides for removing the now capture leaf assembly retained specimen from the patient and, as represented at arrow 294 and block 296, the biopsy specimen is retrieved from the capture component.

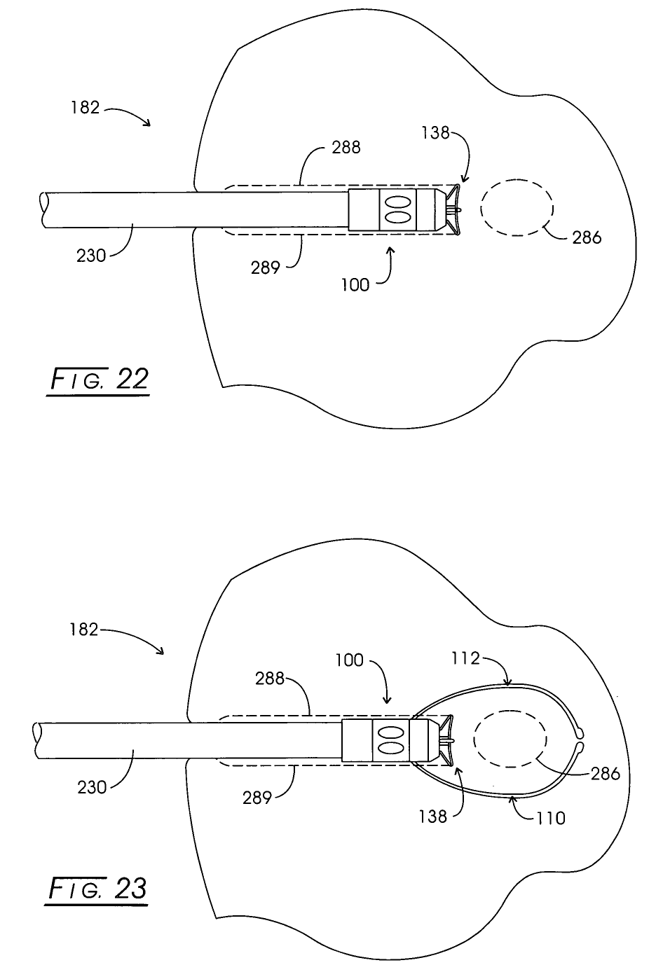

Looking to FIG. 23, the sequence represented at block 292 is schematically portrayed. In this regard, the five leaf capture component structure with leading edge pursing and cutting cables cuts around the target tissue 286 and the pursing action brings the blade tips mutually together to complete severance of the target volume and surrounding healthy tissue. Then, as schematically represented in FIG. 24, the practitioner commences the pulling back and removal of the captured specimen. The figure schematically reveals at dashed boundary 298 a somewhat enlarged region of cut tissue resulting from the activity of the cutting and pursing cables and associated leafs. As the instrument delivery cannula 230 is withdrawn from the initial incision, the resilient leaf structures will tend to compress within the channel established by the earlier energized precursor electrodes.

An advantageous aspect of the recovery instrument 222 resides in its capability for being configured to alter the size and shape of a profile defined by the leafs and associated pursing and cutting cables without enlarging the diameter of the delivery cannula 230. However, as the target tissue volume increases in size, for example to about 20 mm, the outboard or radial extent of the precursor electrodes may be increased. Contact of the radially enlarged precursor electrodes with the precursor cables and/or leafs during the capturing process is not detrimental, the thin flexible precursor electrodes simply flexing axially inwardly.

Referring to FIG. 25, an enlarged target tissue volume is represented schematically in dashed form at 300 in conjunction with tissue 302. Delivery cannula 230 is seen to be in confronting adjacency with target tissue volume 300 within tissue 302. The delivery cannula 230 has been manipulated through skin 304 and its precursor electrode assembly, represented generally at 306, will have made four electrosurgical cuts as represented by the dashed channel boundaries 308 and 309. Note that these boundaries commence respectively at 310 and 311, a location sufficiently subcutaneous to avoid thermal damage outwardly towards skin 304. The precursor electrode assemblage 306 again, preferably, is arranged with four symmetrically radially disposed tissue confronting and cutting surfaces, i.e., in quadrature.

Looking to FIG. 26, a capture of the target tissue volume 300 and surrounding healthy tissue is schematically represented by the leaf profiles 314 and 316. Precursor electrode assemblies 306 will have been de-energized as this procedure takes place however, the precursor electrodes may be secondarily energized from the pursing/cutting cables associated with the leafs with beneficial effect during the deployment of the leafs.

Looking to FIG. 28, withdraw of the retrieval instrument is schematically portrayed. In the figure, the profile of the now excised tissue is represented at dashed boundary 318. As before, during removal, the leaf structures represented schematically at 314 and 316 will tend to compress along with the captured tissue volume. Tissue flexure permits removal and, again, the advantage of the flexibility of the precursor electrode 306 assembly becomes apparent.

Since certain changes may be made in the above-described apparatus and method without departing from the scope of the invention herein involved, it is intended that all matter contained in the description thereof or shown in the accompanying drawings shall be interpreted as illustrative and not in a limiting sense.

1. An insertion instrument for aiding the positioning of a tip of a cannular instrument, with a surface having a given cross-sectional dimension, at a select depth within an incision of predetermined length extending along the skin of a patient, said tip carrying a forwardly disposed energizable cutting component, comprising:
a first retractor component movable along a first retraction locus, said first retractor component having an outwardly disposed first tissue engagement surface extending along a first axis from a first tip, of first insertion entry dimension, an insertion depth length corresponding with said select depth to a first insertion position with a dimension generally transverse to said first axis of extent generally corresponding with said given cross-sectional dimension of said instrument surface, and having an oppositely disposed generally concave first instrument guide surface at least a portion of which is contoured in correspondence with a first portion of said instrument surface an amount effective to engage said instrument in generally guiding slidable relationship;
a second retractor component movable along a second retraction locus generally aligned with and oppositely directed from said first retraction locus, said second retractor component having an outwardly disposed second tissue engagement surface extending along a second axis from a second tip, of second insertion entry dimension, said insertion depth length, corresponding with said select depth, to a second insertion position with a dimension generally transverse to said second axis of extent generally corresponding with said given cross-sectional dimension of said instrument surface, and having an oppositely disposed generally concave second instrument guide surface contoured in correspondence with a second portion of said instrument surface opposite said first portion an amount effective to engage said instrument in generally guiding slidable relationship; and
at least one of said first and second retractor components being movable along respective said first and second retraction loci toward and away from an initial orientation of an adjacency of said instrument guide first and second surfaces.

2. The insertion instrument of claim 1 further comprising:
a retractor drive assembly coupled in driving relationship with said first and second retractor components and actuable to cause said first retractor component to move along said first retraction locus from said initial orientation.

3. The insertion instrument of claim 2 in which said retractor drive assembly is actuable to cause said second retractor component to move along said second retraction locus from said initial orientation.

4. The insertion instrument of claim 2 in which:
said first retractor component includes a first threshold portion coupled with said retractor drive assembly, and integrally formed with and configured as an extension of said first instrument guide surface and outwardly tapering to define a first entrance mouth portion; and
said second retractor component includes a second threshold portion coupled with said retractor drive assembly and integrally formed and configured as an extension of said second instrument guide surface and outwardly tapering to define a second entrance mouth portion.

5. The insertion instrument of claim 1 in which:
said first retractor component outwardly disposed first tissue engagement surface is outwardly convexly-shaped and tapered toward said first tip; and
said second retractor component second tissue engagement surface is outwardly convexly-shaped and tapered toward said second tip.

6. The insertion instrument of claim 1 in which:
said first retractor component first tissue engagement surface is configured with discontinuities effective to enhance an engagement with tissue within said incision; and
said second retractor component second tissue engagement surface is configured with discontinuities effective to enhance an engagement with tissue within said incision.

7. The insertion instrument of claim 6 in which:
said first retractor component first tissue engagement surface discontinuities comprise grooves; and
said second retractor component second tissue engagement surface discontinuities comprise grooves.

8. The insertion instrument of claim 7 in which:
said first retractor component first tissue engagement surface grooves are disposed in mutually parallel relationship generally normally to said first axis; and
said second retractor component second tissue engagement surface grooves are disposed in mutually parallel relationship generally normally to said second axis.

9. The insertion instrument of claim 1 in which:
said first retractor component first tissue engagement surface includes a visible indicia spaced from said first tip a length corresponding with said select depth.

10. The insertion instrument of claim 9 in which:
said second retractor component second tissue engagement surface includes a visible indicia spaced from said second tip a length corresponding with said select depth.

11. The insertion instrument of claim 2 in which said retractor drive assembly comprises:
a first lever coupled with said first retractor component, extending therefrom a pivot distance to a first pivot location, and extending from said first pivot location to define a manually graspable first handle component;
a second lever coupled with said second retractor component, extending therefrom said pivot distance to a second pivot location, and extending from said second pivot location to define a manually graspable second handle component;
a connector assembly pivotally connecting said first lever first pivot location with said second lever second pivot location; and
a spring assembly coupled in mutually outwardly biasing relationship intermediate said first handle component and said second handle component.

12. The insertion instrument of claim 11 in which said retractor drive assembly further comprises:
a stop member extending from said first handle component toward said second handle component a distance selected to limit the extent of separation of said first retractor component from said second retractor component when said first and second handle components are manually urged toward each other.

13. The method for accessing a target tissue volume of predetermined diametric extent located beneath the skin of a patient with the tip of a cannular instrument having a given cross-sectional dimension and carrying a forwardly disposed energizable cutting assembly, comprising the steps of:
(a) determining an instrument entry location upon said skin;
(b) making an incision through said skin at said entry location having an incision length at least corresponding with said cross-sectional dimension and an incision depth effective to avoid thermal damage to the dermis of said skin when said cutting electrode assembly is energized;
(c) providing a retractor assembly having first and second retractor components each having an outwardly disposed engagement surface extending from a retractor tip an insertion entry length corresponding with said incision depth to an insertion position and having mutually inwardly disposed generally concave and generally cylindrically-shaped instrument guide surfaces, and a retractor drive assembly actuable to move at least one said first and second retractor components from an initial mutually abutting position to oppositely disposed spaced apart retracting positions;
(d) inserting said first and second retractor component tips within said incision to locate said incision position at the surface of said skin;
(e) actuating said retractor drive assembly to move at least one said first and second retractor components to said spaced apart retracting positions wherein said instrument guide surfaces are mutually spaced apart a distance at least corresponding with said instrument given cross-sectional dimension to define a guidance channel;
(f) inserting said instrument tip along said guidance channel to a position adjacent said first and second retractor component tips;
(g) removing said first and second retractor components from said incision;
(h) applying cutting energy to said cutting assembly; and
(i) positioning said instrument tip into confronting adjacency with said target tissue volume.

14. The method of claim 13 in which said step (b) makes said incision of a said length greater than said cross-sectional dimension and less than a length corresponding with said target tissue volume predetermined diametric extent.

15. The method of claim 13 in which said step (g) of removing said first and second retractor components is carried out before said step (h) of applying cutting energy to said cutting assembly.

16. The method of claim 15 in which said step (i) of positioning said instrument tip is carried out while applying cutting energy to said cutting assembly.

17. The method of claim 13 in which said step (c) provides said retractor assembly with a said retractor drive assembly which is configured for causing said first retractor component to move simultaneously and at the same rate of movement as said second retractor component, when actuated.

18. The method of claim 13 in which said step (c) provides said first retractor component as including a first threshold portion coupled with said retractor drive assembly and integrally formed with and configured as an extension of said instrument guide surface and outwardly tapering to define a first entrance mouth portion, and providing said second retractor component as including a second threshold portion coupled with said retractor drive assembly and integrally formed and configured as an extension of said instrument guide surface and outwardly tapering to define a second entrance mouth portion.

19. The method of claim 13 in which said step (c) provides each said first and second retractor component as being outwardly convexly shaped and tapered toward said tip.

20. The method of claim 13 in which:
said step (c) provides each said first and second retractor component tissue engagement surface as being configured with discontinuities effective to enhance an engagement with tissue within said incision.

21. The method of claim 20 in which said step (d) is carried out while drawing said tissue within said incision outwardly about said instrument tip.

22. The method of claim 20 in which said discontinuities are provided as grooves.

23. The method of claim 13 in which:
said step (c) provides said first retractor component tissue engagement surface as having a visible indicia spaced from said tip thereof a length corresponding with said incision depth; and
said step (d) carries out said insertion by inserting said first and second retractor components within said incision until the surface of said skin adjacent said incision is aligned with said visible indicia.

24. The method of claim 23 in which said step (c) provides said second retractor component tissue engagement surface as having a visible indicia spaced from said tip thereof a length corresponding with said incision depth.

25. The method of claim 13 in which:
said step (c) provides said retractor drive assembly as comprising:
a first lever coupled with said first retractor component, extending therefrom a pivot distance to a first pivot location, and extending from said first pivot location to define a manually graspable first handle component,
a second lever coupled with said second retractor component, extending therefrom said pivot distance to a second pivot location, and extending from said second pivot location to define a manually graspable second handle component,
a connector assembly pivotally connecting said first lever first pivot location with said second lever second pivot location, and
a spring assembly coupled in mutually outwardly biasing relationship intermediate said first handle component and said second handle component; and
said step (e) carries out said actuation of said retractor drive assembly by urging said first and second handle components towards each other.

26. The method of claim 25 in which:
said retractor drive assembly is provided as further comprising a stop member extending from said first handle component toward said second handle component a distance selected to limit the extent of separation of said first retractor component from said second retractor component when said first and second handle components are urged toward each other.

27. An electrosurgical instrument comprising:
a support member having a central axis and a surface of given radius, diameter and circumference and having a forwardly disposed tip surface, said support member being configured for movement through tissue towards a select location within the body of a patent;
an energizable cutting electrode assembly having at least three thin electrode branch portions energizable to cut said tissue, arranged generally normally to and generally symmetrically about said central axis, said branch portions being located forwardly from said tip surface a spacing distance effective to enhance the forward displacement of said tissue, when cut, to facilitate a slideable engagement of said cut tissue with said support member surface; and
a source actuable to apply cutting energy to said electrode branch portions.

28. The electrosurgical instrument of claim 27 in which:
said thin electrode branch portions extend along radii disposed outwardly from said central axis.

29. The electrosurgical instrument of claim 27 in which:
each said electrode branch portion includes a tissue confronting and cutting surface generally located within a common plane disposed normally to said central axis.

30. The electrosurgical instrument of claim 27 in which each said electrode branch portion is fixed with respect to said support member tip surface.

31. The electrosurgical instrument of claim 30 in which each said electrode branch portion extends outwardly from said central axis an extent to be at least co-extensive with said support member surface circumference.

32. The electrosurgical instrument of claim 27 in which said electrosurgical electrode assembly is configured to cut said tissue with a formation of an equivalent cut diameter of at least about 90% of said support member diameter.

33. The electrosurgical instrument of claim 27 in which four said electrode branch portions are provided having tissue confronting and cuffing surfaces arranged symmetrically about said central axis.

34. The electrosurgical instrument of claim 33 in which each said electrode branch portion extends outwardly from said central axis an extent to be at least co-extensive with said support member surface circumference.

35. The electrosurgical instrument of claim 33 in which each said electrode branch portion is fixed with respect to said support member tip surface.

36. The electrosurgical instrument of claim 33 in which each of said four electrode branch portion confronting and cutting surfaces is generally located in a common plane disposed normally to said central axis.

37. The electrosurgical instrument of claim 27 in which:
said energizable cutting electrode assembly branch portions include tissue confronting and cutting surfaces generally located within a common plane disposed normally to said central axis; and
said spacing distance is within a range from about 0.5 mm to about 5 mm.

38. The electrosurgical instrument of claim 27 in which:
said energizable cutting electrode assembly branch portions include tissue confronting and cutting surfaces generally located within a common plane disposed normally to said central axis; and
said spacing distance is within a range from about 1 mm to about 2 mm.

39. The method for accessing a target tissue volume of predetermined diametric extent located beneath the skin of a patient, comprising the steps of:
(a) providing a recovery instrument having a cannular support member with a central axis, a surface of given instrument radius and diameter extending to a tip surface, having a cutting electrode assembly with at least three thin, flexible electrode branch portions energizable to cut tissue, arranged generally normally to and generally symmetrically about said central axis, and having a radial extent co-extensive with said given radius and exhibiting an equivalent diameter of at least about 90% of said instrument diameter;
(b) determining an instrument entry location upon said skin;
(c) making an incision through said skin at said entry location having an incision length at least corresponding with said support member diameter and an incision depth effective to avoid thermal damage to the dermis of said skin when said cutting electrode assembly is energized;
(d) providing a retractor assembly having first and second retractor components each having an outwardly disposed engagement surface extending from a retractor tip an insertion entry length corresponding with said incision depth to an insertion position and having mutually inwardly disposed generally concave and generally cylindrically-shaped instrument guide surfaces, and a retractor drive assembly actuable to move at least one said first and second retractor components from an initial mutually abutting position to oppositely disposed spaced apart retracting positions;
(e) inserting said first and second retractor component tips within said incision to locate said incision position at the surface of said skin;
(f) actuating said retractor drive assembly to move at least one said first and second retractor components to said spaced apart retracting positions wherein said instrument guide surfaces are mutually spaced apart a distance at least corresponding with said instrument support member diameter to define a guidance channel;
(g) advancing said recovery instrument tip within said guidance channel to an extent locating said electrode branch portions adjacent said retractor tip;
(h) removing said first and second retractor components from said incision;
(i) applying cutting energy to said cutting electrode assembly; and
(j) advancing said recovery instrument and tip surface into confronting adjacency with said target tissue volume.

40. The method of claim 39 in which said step (a) provides said recovery instrument having said cutting electrode assembly branch portions located forwardly from said tip surface a spacing distance effective to enhance the forward displacement of tissue when said step A) advancing said recovery instrument is carried out.

41. The method of claim 40 in which said step (a) provides said recovery instrument as having a said cutting electrode assembly with said branch portions having tissue confronting and cutting surfaces generally located within a common plane disposed normally to said central axis.

42. The method of claim 39 in which said step (a) provides said recovery instrument as having four said cutting electrode branch portions arranged symmetrically about said central axis.

43. The method of claim 41 in which said step (a) provides said recovery instrument as having four said cutting electrode branch portions arranged symmetrically about said central axis.
